(19) United States
(12) Patent Application Publication
(10) Pub. No.: US 2026/0033977 A1
(43) Pub. Date: Feb. 5, 2026

(54) NAIL CORRECTION KIT

(71) Applicant: NEUBOURG SKIN CARE GMBH, GREVEN (DE)

(72) Inventor: Diana HOEJBERG, Risskov (DK)

(21) Appl. No.: 19/357,770

(22) Filed: Oct. 14, 2025

Related U.S. Application Data
(63) Continuation of application No. 18/092,568, filed on Jan. 3, 2023, now Pat. No. 12,458,523.
Application No. 18/092,568 is a division of application No. 16/612,845, now abandoned.

(30) Foreign Application Priority Data
May 12, 2017 (DE) 10 2017 004 546.1

(51) Int. Cl. A61F 5/11

(52) U.S. Cl. CPC A61F 5/11

(57) ABSTRACT

The present invention relates to a kit for performing a nail correction method for a human or animal toenail or fingernail.

### CROSS-REFERENCE TO RELATED APPLICATION

This application is a continuation of U.S. Ser. No. 18/092,568, filed Jan. 3, 2023, which is a divisional of U.S. Ser. No. 16/612,845, filed Nov. 12, 2019, which is a 371 National Phase of International Application No. PCT/IB2018/053356, filed May 14, 2018, which claims the benefit of DE 10 2017 004 546.1, filed May 12, 2017, the contents of which are incorporated herein by reference.

### BACKGROUND OF THE INVENTION

The present invention relates to a kit for performing a nail correction method for a human or animal toenail or fingernail. The application claims priority of the prior application DE 102017004546.1 (date of filing: 12 May 2017).

Various methods are known to correct toenails or fingernails. Such nails frequently have a strong curvature causing them to press or grow into the nail bed. A common way of treatment is to remove the complete nail by surgery. The treatment is not only painful, but often does not result in a nail correction: The newly growing nail is frequently as curved as the original one nail was.

An alternative, non-surgical way of treatment is to fit a nail correction brace at the respective nail. Such a brace is described in the German patent specification DE 10 2005 039 147 B3. The brace engages under the nail, which often will lead to tissue irritations. In WO 2011/039243 A1, a multi-layer polymer system is described, wherein it is intended that, by contraction and expansion of different layers in opposite directions, lateral tensile forces on the nail are obtained, with the consequence of a mechanical correction. As has been found, the method described in WO 2011/039243 A1 is not reproducible: All efforts to reproduce such a multi-layer system, which, on the one hand, attaches on the nail and, on the other hand, develops the required correction forces, were not successful.

There is, therefore, a continuing need for means for the correction of misgrown toenails or fingernails.

### SUMMARY OF THE INVENTION

Surprisingly, it has been found that the kit for nail correction described in the following avoids the above drawbacks and enables in a simple way a correction of ingrown fingernails or toenails. Depending on the degree of deformation of the nails (in particular on the degree of curvature), different compositions based on photopolymerizable components can be applied. Optionally, one or more metallic spring braces can additionally be used. By the kind of attachment of the metallic spring braces, it can be excluded that mechanical tissue irritations will occur.

The kit for nail correction according to the invention includes
a) a primer, including
40-60% of hydroxyethylene methacrylate,
40-60% of phosphate dimethacrylate,
0.1-1.0% of a starter,
b) at least one composition for making a light-curing nail brace, including
15-45% of bisphenol A (di) methacrylate, urethane dimethacrylate in a proportion from 1:5 to 5:1,
85-55% of fillers and pigments,
0.1-1% of camphorquinone, amino starter, and
c) optionally one or more spring braces.

The component a of the kit according to the invention is a primer, acting as an adhesion agent. During the investigations in connection with the present invention, it has been found that the adhesion of the photopolymer to the nail is critical for the success of the correction method. It turns out that the adhesion of the photopolymer to the nail is significantly improved when the adhesion agent described in the invention is employed as a primer. This adhesion agent includes 40-60% of hydroxyethylene methacrylate and 40-60% of phosphate dimethacrylate and 0.1-1.0% of a polymerization starter. The polymerization starters are described below in more detail. The mixing proportion may vary within the percentages mentioned above. Advantageously, the two methacrylates are included in an approximately identical proportion. It is understood that all components together sum up to 100%.

The actual nail brace is formed by means of the photopolymerizable material b. This is a composition including
15-45% of bisphenol A (di) methacrylate, urethane dimethacrylate in a proportion from 1:5 to 5:1,
   85-55% of fillers and pigments,
   0.1-1% of camphorquinone, amino starter.

The range of compositions mentioned above allows for a different adjustment of the mechanical properties, in the form of different degrees of hardness. In practice, it has proven to provide two compositions, one of which is comparatively soft, the other one being comparatively hard.

The soft composition includes, for instance,
17-21% of bisphenol A (di) methacrylate, urethane dimethacrylate in a proportion from 1:4 to 4:1,
   79-83% of fillers and pigments,
   0.1-1% of camphorquinone, amino starter.

The hard composition includes, for instance,
36-40% of bisphenol A (di) methacrylate, urethane dimethacrylate in a proportion from 1:4 to 4:1,
   60-64% of fillers and pigments,
   0.1-1% of camphorquinone, amino starter.

The monomers bisphenol A (di) methacrylate and urethane dimethacrylate included in the compositions are preferably included in approximately equal amounts. Mixing proportions, wherein the two main components are included in a mass proportion between 1:5 and 5:1, are generally suitable. Particularly preferable are mixing proportions, wherein the two main components are included in a mass proportion between 1:2 and 2:1.

The material for the light-curing nail brace further includes fillers and pigments. These are silicate-based fillers (silica fillers), fillers based on ground barium glass (barium glass fillers) as well as polymeric particles. The particular additions have diameters in the range from 0.1-10 μm, preferably they are smaller than 5 μm. In particular, the barium glass-based fillers significantly contribute to the mechanical properties of the nail brace. In addition, pigments may further be included in the composition, in order to confer an aesthetic appearance to the formed nail brace. Further, the pigments should preferably have diameters in the range from 0.1-10 μm.

The compositions according to the invention further include polymerization starters that can initiate the desired polymerization reaction by irradiation with light. For this purpose, in principle, all classical polymerization starters are suitable. Particularly suitable has proven the combination of camphorquinone with amino starters, namely tertiary amines (e.g., triethanolamine, N,N-dimethyl-p-toluidine, triethylamine, 4-dimethylamino bezoic acid ethyl ester, N,N-tetramethylaniline). Alternatively, for instance, 2-ethylanthraquinone in combination with N-phenylglycine can be used. When using such a system, surprisingly, the completed composition can be stored over longer times, without a polymerization reaction taking place. Only after illumination with an adjusted light source, the polymerization reaction will occur. For this purpose, e.g., an LED light source with a wavelength of approx. 450 nm and a light power of 1000-1500 mW/cm2 can be used.

Optionally, the kit according to the invention further includes one or more braces made of spring steel. They are made from a spring steel wire having a diameter of 0.3-1.5 mm. The brace may include one or more U-shaped loops. The metal spring brace is applied with the light-curing material described above on top of the respective nail (glued). Different from the therapy methods described above using such metal braces, it is neither required to enclose the nail at the outside, nor to provide for boreholes in the nail. FIG. 4 shows an attachment according to the invention for such a metal brace.

### BRIEF DESCRIPTION OF THE DRAWINGS

FIG. 1 shows the application of a composition for making a light-curing nail brace onto the nail.

FIG. 2 shows the light-cured composition on the nail close to the nail pocket (after illumination).

FIG. 3 shows the application of a light-curing composition on the nail close to the nail pocket.

FIG. 4 shows the attachment of a spring brace by means of the kit according to the invention by fixing the first end of the spring brace (top) and then fixing the other end.

### DETAILED DESCRIPTION

The kit according to the invention is used as follows:

First, the therapist removes the concerned nail from the nail bed. In this procedure, a strip of cloth can be inserted between nail and nail bed. Then, the nail is first treated with the primer of the kit. Care has, in particular, to be taken that the nail is dry. The concerned nail should, in particular during the 24 hours before the application, not have been in a longer-lasting contact with water. The patient should, for instance, not have taken a bath. Short washing or showering is, however, harmless, provided that the nail has been carefully dried. If necessary, the nail can be dried with a hot-air blower. The primer is first applied onto the necessary locations. Normally, it is recommended to apply the primer in a sheet-like manner onto the entire nail. After application, the polymerization is started by means of a light source (preferably blue light of approx. 450 nm and 100 mW/cm²). When using a usual light source, the polymerization is completed after a time of 5 seconds to 60 seconds, normally 10 seconds illumination is sufficient.

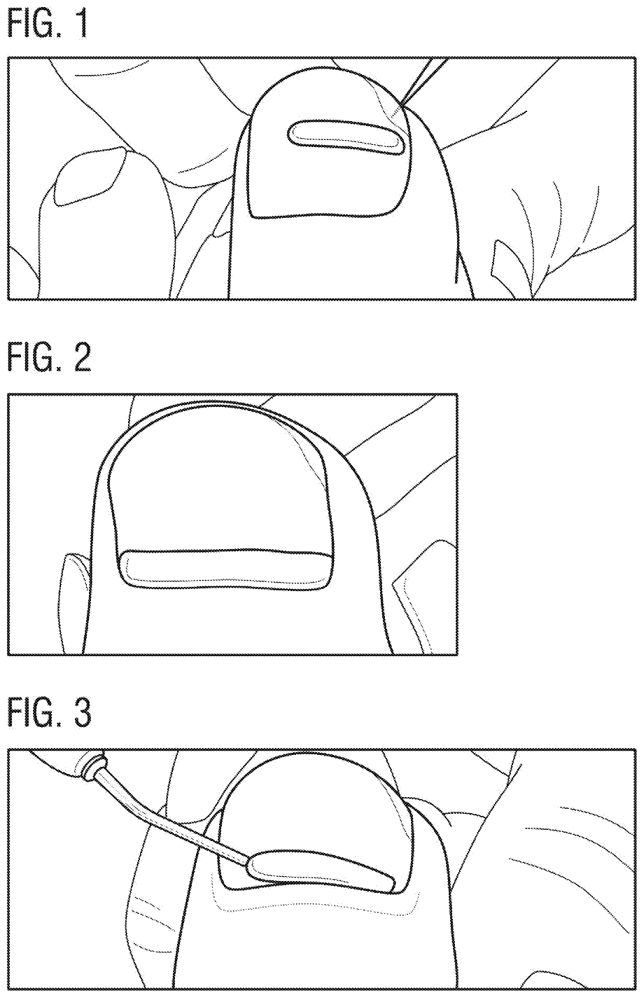

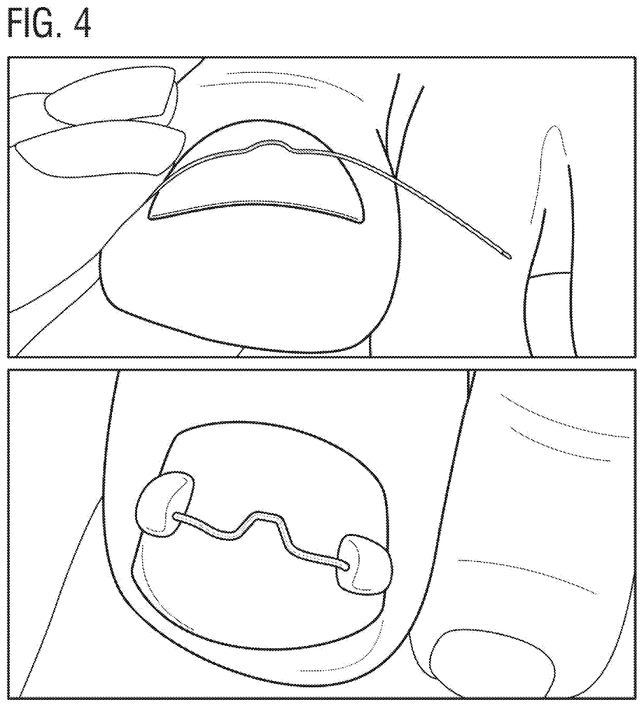

Thereafter, the light-curing nail brace is applied. The nail is held by the therapist by means of a tool in the desired position. The application is made in the form of lines (see FIG. 1) with a line width of 2-6 mm. After the application, preferably immediately, the polymerization is started by illumination with the light source mentioned above. It is important, when doing so, to hold the nail in the desired shape. This step, too, is usually completed after a time of 5-60 seconds. Thereafter, the material can again be re-ground, so that no edges will be created, where fabric (e.g., stockings) could get caught. With strongly deformed or very strongly thickened nails, a multitude of such line-shaped applications can be performed. If applicable, very thick nails may also be ground preparatorily, so that the nail is deformable again. Grinding has, of course, to be performed before the priming process with the primer. The photopolymer may also be applied in a correspondingly thicker layer. In an extreme case, a metallic spring brace may be applied. For this purpose, first, a drop of the light-curing material is applied as a point, and the spring brace is pressed into this drop (FIG. 4). After light-curing of the drop, the spring brace is spanned over the nail and fixed by means of a second drop of the photopolymerizing material. The two ends of the metal brace should each be visible from the inside of the drop. In this embodiment, too, a preparation of the nail using the primer is required, since otherwise the durability of the construct on the nail cannot be guaranteed.

By means of the kit according to the invention, it is also possible to enable another correction of a nail already treated with a metal brace. In particular, in the case of a tissue irritation (nail bed irritation) by the classical metal brace, the classical metal brace can be removed, and the nail is then further treated with the kit according to the invention.

The compositions provided in the kit are preferably supplied in correspondingly designed containers. For the primer, in principle, vials made of glass or plastic with an application brush are suitable. The compositions for making light-curing nail braces are typically more viscous and are preferably supplied in cartridges for use together with a cartridge press or gun. All containers are preferably opaque.

With the kit according to the invention, the necessary materials are provided, in order to correct ingrown toenails or fingernails for humans or animals, without the drawbacks of prior art occurring. In most cases, the nail correction can be performed without the aid of metallic braces. The composition according to the invention, in particular, guarantees a clearly better adhesion to the nail than prior art compositions. Furthermore, the advantages of a polymer can be combined with those of a metal brace, without the drawbacks occurring that frequently appeared in prior metal braces, in particular mechanical irritations of the nail bed.

In an improvement of the composition according to the invention, the primer additionally includes one or more antimicotics. As has been found in practice, nails needing a correction are frequently infested by nail fungi causing additional problems. It is known that nail fungi cannot easily be treated. It turned out that the nail fungus treatment is successful, when the primer additionally includes one or more antimicotics. The admixture is normally 0.1-2%, preferably 0.5-1%. As an antimcotic, generally compounds are suitable that are approved for the treatment of onychomycoses, such as, for instance, econazole, bifonazole, chlodrimazole, fenticonazole, ketocanazole, miconazole, oxiconazole, and related compounds.

In the embodiment of the invention with an addition of antimycotics, the complementing addition of penetration amplifiers has proven successful. For this purpose, the usual penetration amplifiers for nail penetration can be used. Particularly successful for the present invention have proven penetration amplifiers based on substituted 1,3-dioxolanes, 1,5-dioxanes and acetals, in particular the substances and substance mixtures marketed under the trademark SEPA®.

### Examples

The invention is further explained by the compositions exemplarily illustrated in the following:

### A) Primer

| | A1 | A2 | A3 | A4 |
|---|---|---|---|---|
| Component | (wt.-%) | (wt.-%) | (wt.-%) | (wt.-%) |
| Hydroxyethylene | 49.7 | 39.7 | 35.7 | 45.7 |
| methacrylate | | | | |
| Phosphate | 49.7 | 59.7 | 54.6 | 44.7 |
| dimethacrylate | | | | |
| [bis(glyceryl- | | | | |
| dimethacrylate) | | | | |
| phosphate] | | | | |
| Camphorquinone | 0.4 | 0.4 | 0.5 | 0.4 |
| Triethylamine | 0.2 | | 0.1 | 0.2 |
| N,N-Dimethyl-p- | | 0.2 | 0.1 | |
| toluidine | | | | |

### B) Nail Brace (Soft)

| | B1 | B2 | B3 | B4 | B5 |
|---|---|---|---|---|---|
| Component | (wt.-%) | (wt.-%) | (wt.-%) | (wt.-%) | (wt.-%) |
| Bisphenol A | 16.0 | 15.0 | 14.0 | 30.0 | 22.0 |
| (di)methacrylate | | | | | |
| Urethane | 16.0 | 30.0 | 30.0 | 15.0 | 22.0 |
| dimethacrylate | | | | | |
| Silica filler | 20.0 | 0.0 | 17.0 | 16.5 | 18.5 |
| (Aerosil 9200) | | | | | |
| Silica filler | 5.0 | 10.3 | 15.0 | 12.5 | 4.5 |
| (Aerosil 7200) | | | | | |
| Barium glass (median | 18.0 | 13.0 | 13.0 | 12.0 | 14.5.0 |
| particle size: 13 μm) | | | | | |
| Barium glass (median | 3.9 | 14.0 | 5.2 | 5.0 | 5.5 |
| particle size: 5 μm) | | | | | |
| Polymeric particle | 20.0 | 12.0 | 4.0 | 4.5 | 8.0 |
| (median particle | | | | | |
| size: 10 μm) | | | | | |
| Polymeric particle | 0.0 | 5.0 | 1.0 | 3.5 | 4.0 |
| (median particle | | | | | |
| size: 6 μm) | | | | | |
| Camphorquinone | 0.6 | 0.4 | 0.5 | 0.6 | 0.0 |
| Triethylamine | 0.5 | 0.0 | 0.1 | 0.4 | 0.0 |
| N,N-Dimethyl-p- | 0.0 | 0.3 | 0.2 | 0.0 | 0.0 |
| toluidine | | | | | |
| 2-Ethylanthraquinone | 0.0 | 0.0 | 0.0 | 0.0 | 0.6 |
| N-Phenylglycine | 0.0 | 0.0 | 0.0 | 0.0 | 0.4 |

### C) Nail Brace (Hard)

| | C1 | C2 | C3 | C4 | C5 |
|---|---|---|---|---|---|
| Component | (wt.-%) | (wt.-%) | (wt.-%) | (wt.-%) | (wt.-%) |
| Bisphenol A | 20.0 | 25.0 | 18.0 | 19.0 | 15.0 |
| (di)methacrylate | | | | | |
| Urethane | 20.0 | 13.0 | 20.0 | 20.0 | 23.0 |
| dimethacrylate | | | | | |
| Silica filler | 20.0 | 0.0 | 18.0 | 16.5 | 19.5 |
| (Aerosil 9200) | | | | | |
| Silica filler | 5.0 | 13.3 | 16.0 | 15.5 | 5.5 |
| (Aerosil 7200) | | | | | |
| Barium glass (median | 16.0 | 14.0 | 12.5 | 12.0 | 12.5 |
| particle size: 13 μm) | | | | | |
| Barium glass (median | 3.9 | 16.1 | 7.0 | 6.5 | 8.5 |
| particle size: 5 μm) | | | | | |
| Polymeric particle | 14.0 | 12.0 | 6.2 | 6.0 | 11.0 |
| (median particle | | | | | |
| size: 10 μm) | | | | | |
| Polymeric particle | 0.0 | 5.0 | 1.0 | 3.5 | 4.0 |
| (median particle | | | | | |
| size: 6 μm) | | | | | |
| Camphorquinone | 0.6 | 0.8 | 0.7 | 0.6 | 0.0 |
| Triethylamine | 0.5 | 0.0 | 0.2 | 0.4 | 0.0 |
| N,N-Dimethyl-p- | 0.0 | 0.8 | 0.4 | 0.0 | 0.0 |
| toluidine | | | | | |
| 2-Ethylanthraquinone | 0.0 | 0.0 | 0.0 | 0.0 | 0.6 |
| N-Phenylglycine | 0.0 | 0.0 | 0.0 | 0.0 | 0.4 |

What is claimed is:

1. A method of nail correction for a human or animal fingernail or toenail, comprising the steps of:

2. The method according to claim 1, wherein the primer comprises 40 to 60 percent by weight of hydroxyethylene methacrylate, 40 to 60 percent by weight of phosphate dimethacrylate, and 0.1 to 1.0 percent by weight of a polymerization starter.

3. The method according to claim 2, wherein the polymerization starter comprises camphorquinone and an amino starter.

4. The method according to claim 1, wherein the light-curing composition comprises 15 to 45 percent by weight of bisphenol A dimethacrylate and urethane dimethacrylate in a mass ratio from 1:5 to 5:1, 85 to 55 percent by weight of fillers and pigments, and 0.1 to 1 percent by weight of camphorquinone and an amino starter.

5. The method according to claim 4, wherein the fillers comprise at least one filler selected from silicate-based fillers, barium glass fillers, and polymeric particles.

6. The method according to claim 4, wherein the pigments and fillers have particle sizes in the range of 0.1 to 10 micrometers.

7. The method according to claim 1, wherein the primer is applied in a sheet-like manner over the entire nail surface.

8. The method according to claim 1, wherein the light-curing composition is applied in multiple line-shaped applications.

9. The method according to claim 1, wherein the light-curing composition is applied in a thickness sufficient to deform a thickened nail.

10. The method according to claim 1, wherein the light-curing composition is re-ground after curing to remove any sharp edges.

11. The method according to claim 1, further comprising applying a spring brace to the nail using the light-curing composition.

12. The method according to claim 11, wherein the spring brace is fixed by pressing it into a first portion of the light-curing composition, curing said first portion, spanning the spring brace over the nail, and fixing the opposite end using a second portion of the light-curing composition.

13. The method according to claim 1, wherein the primer further comprises 0.1 to 2 percent by weight of one or more antimycotic agents.

14. The method according to claim 13, wherein the primer further comprises one or more penetration amplifiers based on substituted 1,3-dioxolanes, 1,5-dioxanes, or acetals.

15. The method according to claim 1, wherein the polymerization of the primer is initiated using a light source having a wavelength of approximately 450 nanometers.

16. The method according to claim 15, wherein the light source has an intensity in the range of 100 to 1500 milliwatts per square centimeter.

17. The method according to claim 1, wherein the primer is polymerized by irradiating with light for a duration of 5 to 60 seconds.

18. The method according to claim 17, wherein the primer is polymerized by irradiating with light for approximately 10 seconds.

19. The method according to claim 1, wherein the polymerization of the light-curing composition is initiated immediately after application.

20. The method according to claim 1, wherein the light-curing composition is polymerized by irradiating with light for a duration of 5 to 60 seconds.

21. The method according to claim 20, wherein the light-curing composition is polymerized while manually holding the nail in a corrected shape during the irradiation step.

22. The method according to claim 1, wherein the light source is an LED light source.
